(19) United States
(12) Patent Application Publication
(10) Pub. No.: US 2013/0331726 A1
(43) Pub. Date: Dec. 12, 2013

(54) APPARATUS FOR TRACKING COMPLIANCE WITH A TREATMENT FOR OBSTRUCTIVE SLEEP APNEA

(71) Applicant: Claude Weber, Milly la Foret (FR)

(72) Inventor: Claude Weber, Milly la Foret (FR)

(73) Assignee: L'Air Liquide, Société Anonyme pour L'Etude et L'Exploitation des Procédés Georges Claude, Paris (FR)

(21) Appl. No.: 14/001,124

(22) PCT Filed: Jan. 11, 2012
(86) PCT No.: PCT/FR12/50072

§ 371 Date: Aug. 22, 2013

(30) Foreign Application Priority Data
Feb. 24, 2011 (FR) 1151484

(51) Int. Cl. A61B 5/097; A61M 16/06; A61B 5/087; A61M 16/00

(52) U.S. Cl. CPC A61B 5/097; A61M 16/0057; A61M 16/06; A61B 5/087
USPC 600/538; 600/543

(57) ABSTRACT

The invention relates to an apparatus (2, 4) for tracking a treatment for obstructive sleep apnea, including a gas passage (10) and a Venturi tube (16) having a cylindrical inlet (40) and outlet (42), said Venturi tube (16) being axially arranged in said gas passage (10), a first pressure sensor (101), and a second pressure sensor (102). The diameters (D1) of the inlet (40) and outlet (42) of the Venturi tube (16) are between 10 and 25 mm. The Venturi tube (16) includes, arranged in series between said inlet (40) and outlet (42), a convergent portion (44) having the shape of an arc (46, 48), a cylindrical neck (50), the diameter of which is smaller than the diameter (D1) of the inlet (40), and a divergent portion (52) characterized by an angle of divergence (α) of between 5° and 15°. The invention also relates to equipment for treating sleep apnea, which includes a pressurized-gas source (6) connected to a breathing mask (8) via a gas duct (30), as well as to an apparatus (2, 4) according to the invention, which is arranged between said gas source (6) and said breathing mask (8).

### CROSS REFERENCE TO RELATED APPLICATIONS

This application is a 371 of International PCT Application PCT/FR2012/050072, filed Jan. 11, 2012, which claims priority to French Application 1151484, filed Feb. 24, 2011, the entire contents of which are incorporated herein by reference.

### BACKGROUND

The present invention relates to an apparatus for monitoring the compliance with a treatment for obstructive sleep apnea.

Obstructive sleep apnea syndrome (OSAS) is a widespread affliction affecting millions of adults and children, which is characterized by obstruction of the upper airways, which can provoke snoring or breathing stoppages during sleep.

The obstruction which occurs during sleep has two main causes which are lack of muscular tonus and gravity. This is because the excessive presence of tissues in the upper airways and anatomical deformations aggravate the consequences of these factors. Thus, during sleep, particularly during REM sleep, the body relaxes and the muscle tissues, like the tongue and the soft palate for example, lose their rigidity. Moreover, when sleeping is carried out in a stretched-out position, the effect of gravity pushes these tissues toward the bottom of the throat, which closes the upper airways.

When they completely block the upper airways, these tissues prevent the person from breathing at the risk of provoking asphyxia. However, usually, the person wakes up enough to retake control of his or her upper airways and breath, before going back to sleep again. In a person suffering from OSAS, this phenomenon can occur tens or even hundreds of times per night but usually the person has no recollection thereof on awakening.

Now, each obstruction deprives the body of oxygen and therefore forces it to keep carbon dioxide (CO₂) that would normally be expelled in exhalation phases. It follows therefrom that the gas balance of the blood is disrupted and the body is exposed to a “toxic” environment. When the body “signals” that it needs more oxygen, the brain wakes up the sleeper, breathing resumes and the person goes back to sleep again until the next obstruction. These obstructions also lead to an increase in the heart rate and arterial pressure, and possibly lower the “automatic” reaction capability of the body, which is reflected in increasingly severe apneas and hypopneas.

The cyclical micro-awakenings that are experienced by people affected by OSAS affect the quality of their sleep. The symptoms of sleep deprivation in people affected by OSAS are in particular excessive diurnal sleepiness, a lack of concentration, bad memory, or even a depressive state. Hypertension and a lowering of the blood oxygen level are common symptoms in people suffering from sleep apnea, but these are symptoms that are difficult to detect. There are, moreover, other symptoms that are easier to identify such as diurnal sleepiness, snoring, apneas or irregular breathing during sleep, loss of concentration.

An effective OSAS treatment consists in applying a positive air pressure to the airways of the patient. The air pressure acts as an “air cushion” which keeps the upper airways open and prevents the apneas. To do this, use is typically made of an apparatus of CPAP (“Continuous Positive Airway Pressure”) type which delivers slightly pressurized air to the airways of the patient, via a flexible duct, called patient circuit, linked to a breathing mask, generally a nasal or facial mask.

Treatment by CPAP is an effective treatment for patients affected by OSAS if its application is well monitored. It can then lead to a significant improvement in the quality of life of the patient. On the other hand, the effects of the treatment are negligible, even nonexistent, if the patient does not comply with his or her treatment for at least 4 hours per night.

Knowing the compliance of the patients, that is to say measuring the actual time during which they follow their treatment, is therefore essential. Similarly, knowing the effectiveness of the treatment in “real” time is an invaluable aid to adjusting the prescription of the treatment by the treating doctor.

The document WO-A-2009136101 discloses an apparatus for monitoring compliance with a treatment for obstructive sleep apnea comprising a sensor for measuring the air flow rate circulating in the patient circuit.

Inserting such a sensor into the patient circuit can, however, generate a pressure loss which has the effect of reducing the air pressure at the mask of the patient. The actual treatment pressure then no longer corresponds to the prescribed pressure. It is also essential for the sensor to provide a sufficiently reliable signal, while having a limited cost.

The document WO-A-2011/067300 proposes an apparatus for monitoring a breathing parameter of a patient comprising a convergent/divergent device and a pressure measurement system with which to measure the pressure between two portions of said convergent/divergent device having different sections. A similar apparatus is described by US-A-2004/0167419.

Moreover, the document EP-A-2017586 teaches a monitoring device for breathing apparatus of CPAP type comprising a pressure measurement system with which to measure the pressure in a duct conveying pressurized air, said duct passing through said device.

Finally, the international standard ISO 5167-3 lists the characteristics required of the systems for measuring the flow rate of fluids of nozzle and venturi nozzle types used as pressure-reducing apparatus inserted into the ducts of circular section used to convey the fluids.

### SUMMARY

The aim of the present invention is to improve the devices for monitoring the compliance with an oxygen therapy treatment incorporating a venturi tube so as to allow for a measurement that is as accurate and sensitive as possible of the gas flow rate while minimizing the pressure drop resulting from the passage of the gas through the venturi tube.

The solution of the present invention then relates to an apparatus for monitoring the compliance with a treatment for obstructive sleep apnea comprising a gas passage and a venturi tube having a cylindrical inlet and outlet, said venturi tube being arranged axially in said gas passage, a first pressure sensor and a second pressure sensor, characterized in that the diameters D1 of the inlet and the outlet of the venturi tube are between 10 and 25 mm, and the venturi tube comprises, arranged in succession between said inlet and outlet, a convergent portion in the form of a circular arc, a cylindrical neck with a diameter smaller than the diameter D1 of the inlet, and a divergent portion characterized by an angle of divergence α of between 5° and 15°, and in which the diameter D2 of the neck of the venturi tube is between 5 and 15 mm, and the length L2 of the neck of the venturi tube is between 3 and 12 mm.

The venturi tube of the invention makes it possible to perform an accurate and reliable measurement of the flow rate circulating in the gas passage. This is because, knowing that the gas flow rate is proportional to the root of the gas pressure drop between the inlet and the neck of the pressure-reducing apparatus, by virtue of the venturi tube of the invention and of its particular geometry, the pressure drop between the inlet of the venturi tube (sensor) and the neck of the venturi tube is sufficient to obtain a flow rate measurement that is accurate enough to detect breathing events in the patient but, conversely, reliable enough to not affect the treatment of the patient, that is to say no more than 0.2 cm H₂O pressure loss at the outlet for a normal usage of approximately 70 l/min.

The venturi tube is designed to obtain, for the flow rate band considered, a pressure loss less than 80 Pa, and do so by virtue of its particular geometry, in particular the fact of selecting the diameter D2 of the neck to be between 5 and 15 mm and its length L2 to be between 3 and 12 mm, makes it possible to limit the pressure drop in the patient circuit and therefore minimize the impact on the treatment of the patient while retaining a sufficient sensitivity in order to make it possible to measure the compliance with this treatment and detect any residual breathing events.

Using such a venturi tube according to the invention associated with the pressure sensors makes it possible both to obtain a better sensitivity for detecting breathing events and to minimize the disruption to the treatment.

Furthermore, it also offers the advantage of being inexpensive particularly because of the molding possibilities during its manufacture and being insensitive to the moisture content of the air.

Advantageously, the venturi tube of the invention is arranged to limit the pressure loss of the gas between said inlet and outlet, that is to say in such a way as to have a pressure loss, between its inlet and its outlet, less than 100 Pa for a gas flow rate in the passage of between 110 and 150 liters per minute. Preferably, this pressure loss is less than 80 Pa.

According to a preferred embodiment, the venturi tube is of nozzle type. This geometry of the venturi tube conforms to the abovementioned standard SO 5167-3, and offers the advantage of allowing for a low gas pressure loss while ensuring a gas flow stability.

Depending on the case, the apparatus according to the invention may comprise one or more of the following features:
the inlet and the outlet have an identical diameter of between 13 and 20 mm, preferably between 16 and 18 mm.
Advantageously, the inlet and the outlet are cylindrical and have identical diameters D1, preferably between 16 and 18 mm. In fact, the diameter D1 is adapted to the outlet diameter of the air generator of the CPAP treatment apparatus. More preferentially, the diameter D1 of the inlet of the venturi tube is equal to approximately 17.3 mm.
The venturi tube comprises a convergent portion having a profile formed by two successive circular arcs.
The angle of divergence α is less than or equal to 12°, preferably less than or equal to 10°, advantageously equal to approximately 8°.
The diameter D2 of the neck of the venturi tube is between 7 and 13 mm, preferably between 9 and 11 mm, advantageously equal to approximately 10 mm. In fact, the choice of the diameter of the neck and of the angle of divergence allows for a good trade-off between the reduction of the pressure loss in the venturi tube and the increase in its detection sensitivity.
The length L2 of the neck is between 5 and 10 mm, advantageously of the order of 7 to 8 mm, notably equal to approximately 7.5 mm.
The venturi tube comprises a convergent portion having a profile formed by a first circular arc of first radius R1 and a second circular arc of second radius R2 in which said first and second radii are less than 10 mm, preferably less than 5 mm.
The venturi tube has a total length of between 90 and 100 mm.
A first pressure sensor arranged at the inlet of the venturi tube to measure the pressure of the gas at the inlet of the venturi tube; and a second pressure sensor arranged at the neck of the venturi tube to measure the pressure of the gas at the neck of the venturi tube. This enables the gas flow rate to be established.
It comprises a first additional pressure sensor provided at the inlet of the gas passage and measuring the absolute pressure of the gas at the connection with the air generator of the CPAP treatment apparatus and/or a second additional pressure sensor provided at the outlet of the gas passage and measuring the absolute pressure at the connection with the air duct bringing the air to the mask of the patient.
It comprises processing means suitable for processing the flow rate and pressure values measured to deduce therefrom at least one treatment duration datum and at least one treatment effectiveness datum. The processing means may comprise means for correcting any measurement errors due, for example, to the variations of the temperature and/or of the pressure and/or of the humidity of the air.
It comprises data storage means arranged to store at least one of said data.
It comprises transmission means arranged to transmit at least one of said data to a remote server. This remote server is notably situated in the care center in which the patient is monitored or with a service provider allowing the treating doctor remote access to the server. The treating doctor thus has real time data on the compliance with the treatment by the patient.
The data storage means comprise at least one memory chip or one memory card, preferably of plug-in type, for example an SD card or similar.
The transmission means comprise a wireless transmitter system, notably of radio frequency, Bluetooth, Zigbee, wifi, GSM or GPRS type, and an antenna capable of ensuring a wireless transmission of the data that is suited to the type of transmitter and inserted in the module.
The radio-frequency transmitter system comprises a GSM or GPRS modem integrated in or external to the module.
It also comprises one or more indicators, such as colored LEDs, for example red and green, providing the user with information concerning the effectiveness of the treatment.
The venturi tube is arranged in such a way as to have a useful differential pressure approximately 6 times greater than the pressure loss of the gas between the inlet and the outlet of said venturi tube. This ratio between the useful differential pressure and pressure loss of the gas ensures that the venturi tube has a good sensitivity for detecting breathing events.

The invention also relates to a sleep apnea treatment installation comprising a pressurized gas source linked to a breathing mask via a gas duct, characterized in that it comprises an apparatus according to the invention, arranged between said gas source and said breathing mask.

Preferably, the pressurized gas source is a device of CPAP or BiPAP type.

### BRIEF DESCRIPTION OF THE DRAWINGS

Exemplary embodiments of the invention will now by described, in a more detailed but nonlimiting manner, with reference to the appended figures in which:

FIG. 1 is a block diagram illustrating the structure of an apparatus for monitoring compliance according to an embodiment of the invention;

FIG. 2 is a block diagram illustrating the implementation of the apparatus for monitoring compliance of FIG. 1; and

FIG. 3 is a diagram illustrating the geometry of the venturi tube according to an embodiment of the invention.

### DESCRIPTION OF PREFERRED EMBODIMENTS

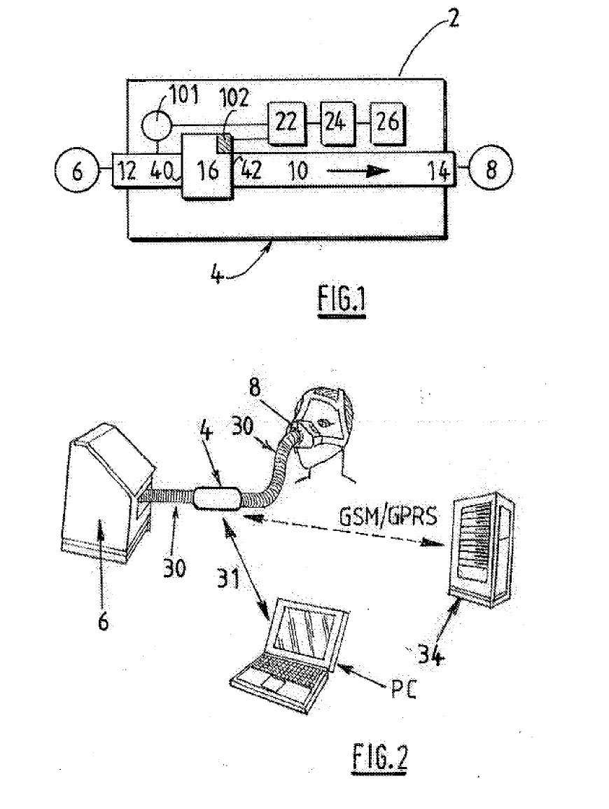

As illustrated in FIG. 2, an apparatus 2, 4 for monitoring the compliance with a treatment for obstructive sleep apnea (OSA) according to the present invention comprises a module 4 that is connected into the path of the breathing gas, typically pressurized air, that is to say on the patient circuit 30 linking an OSA treatment apparatus 6 to the breathing mask 8, generally nasal, with which a patient to be treated is equipped.

The pressure of air delivered by the treatment apparatus 6 is a relative pressure prescribed by the doctor and between 4 and 20 cm H₂O. This pressure is regulated by an air generator of the treatment apparatus 6. It corresponds substantially to the pressure in the mask 8 connected to the nose of the patient to within the tolerance of the pressure drop in the patient's circuit.

The monitoring apparatus 4 is preferably connected to the patient circuit of the treatment apparatus 6 by means of flexible pipes with conventional end fittings, for example end fittings with a diameter equal to 22 mm and conforming to the standard ISO 5356-1.

As illustrated in FIG. 1, the module 4 comprises an internal gas passage 10 with an inlet 12 and an outlet 14 through which passes the gas output by the OSA treatment apparatus 6, before being sent to the patient.

A venturi tube 16 is arranged in the module 4 and linked to the passage 10 so as to allow for a measurement of the flow rate of the gas circulating inside said passage 10, that is to say between the inlet 12 and the outlet 14 of the passage 10. This gas flow rate is notably between 0 and 130 liters per minute. Its walls are, preferably, smooth.

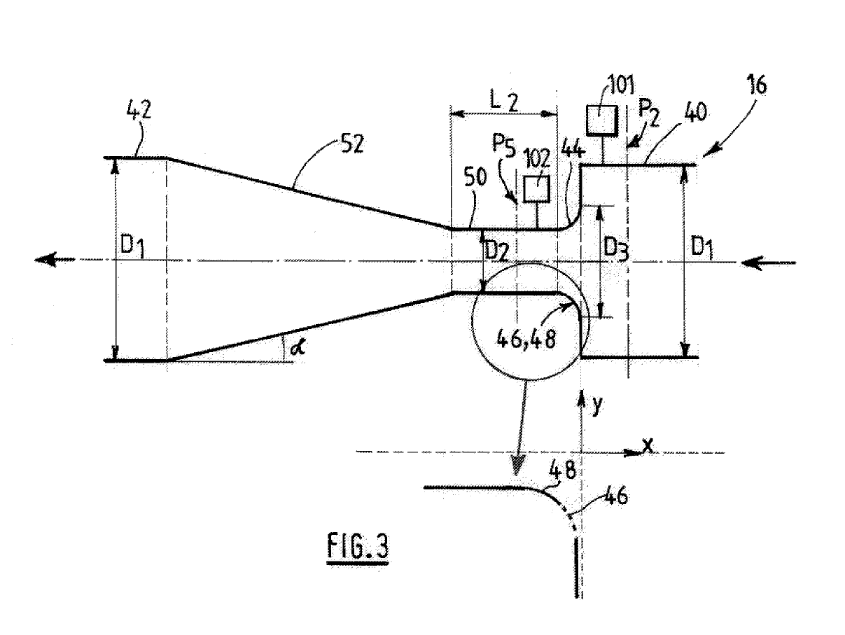

As appears in FIG. 3, the venturi tube 16 comprises two pressure sensors 101, 102. The difference in the pressure measured by the sensors 101 and 102 is proportional to the square of the flow rate in the venturi tube 16.

The pressure sensors 101, 102 of the venturi tube 16 are elsewhere coupled to processing means 22, such as a microcontroller, for example the Texas Instruments MSP430 microcontroller, implementing algorithms, capable of processing the pressure and flow rate measurements in order to deduce therefrom, among other things, the daily treatment duration and the effectiveness of the patient's OSA treatment.

The processing means 22 preferably comprise means for correcting any measurement errors due, for example, to low variation of the temperature and/of the pressure and/or of the humidity of the air.

The pressure sensors 101, 102 can, for example, be BMP085 sensors marketed by the company Bosch, which are high-precision barometric sensors offering an absolute precision up to 0.03 hPa and a consumption as low as 3 μA.

Data storage means 24 are used to store all or part of the duly measured data, for example a data storage memory chip or a plug-in memory card, notably an 8 Gb flash memory card from SST.

Moreover, transmission means 26, for example a radio frequency transmitter and its antenna, are provided to transmit, preferably via a wireless transmission, all or part of said data to a remotely situated receiver, such as a computer or a server, as illustrated in FIG. 2. The radio frequency transmitter may, for example, be equipped with a PHYCOMP 870 MHz antenna.

Electrical current power supply means, not represented, are electrically connected to the sensors 101, 102, to the data storage means 24 and to the transmission means 26 to provide the electrical power supply for the monitoring apparatus 2, for example a low-voltage electrical power supply comprising one or more batteries, cells, etc.

Preferably, the function of the monitoring apparatus 2 is to measure, independently of the OSA treatment apparatus 6, that is to say without using information or data internal to that treatment apparatus 6, and to remotely communicate the information concerning the compliance and effectiveness of the treatment, that is to say the actual patient treatment duration, as well as the events such as apneas, hypopneas, flow rate limitations, snoring, leaks, etc.

This makes it possible to obtain a daily trace and safety in the monitoring of the treatment of the patient in the home by virtue of the possibility of providing an alarm to the patient and his or her care center or the service provider should the prescription not be followed.

As appears in FIG. 2, the module 4 of the compliance monitoring apparatus 2 according to the present invention is incorporated in the path of the gas, that is to say on the duct or ducts 30 conveying gas, and between the treatment apparatus 6 distributing air, notably at continuous positive pressure, and the patient equipped with the nasal mask 8, and makes it possible to measure and record the daily treatment time, as well as the effectiveness of the treatment.

The monitoring apparatus 2 is designed to be able to be adapted to any type of OSAS treatment apparatus 6, that is to say the ventilators of CPAP, BiPAP and similar types.

The monitoring apparatus 2 has a storage capacity of several months, preferably at least one year, which can be further extended.

It can communicate the recorded data via an information transmission link 31, for example radio-frequency RF at a transmission frequency of 868 MHz or 2.4 GHz, or preferably a USB link, to a computer, a PDA, a server or any other means capable of directly recording transmitted data, as shown in FIG. 2. In fact, the monitoring apparatus 2 remotely transmits, using an integrated GSM or GPRS modem, the records of the patient's compliance and treatment effectiveness data to the care center or to the service provider for example, where a suitable server 34 is used to generate treatment compliance and effectiveness reports.

A patient is considered to be well during treatment if two conditions are met, namely:
if the breathing of the patient is detected by a specific algorithm for processing the flow rate and pressure signals measured in the patient circuit making it possible to deduce from these signals that the patient is indeed wearing the nasal or naso-buccal mask; and
if a minimum treatment pressure corresponding to a treatment apparatus that is running and operating correctly is detected.

These two conditions can be determined using pressure and flow rate measurements in the patient circuit by virtue of the monitoring apparatus 2. Such an arrangement makes it possible to deduce the pressure and flow rate variations linked, on the one hand, to the air delivery and, on the other hand, to the inhalations and exhalations of the patient in the patient circuit.

The effectiveness of the treatment is then deduced from the pressure and flow rate variations linked to the inhalations/exhalations of the patient and the treatment pressure level. It is measured by the detection of the number of apneas, hypopneas, flow rate limitations, leaks of the patient circuit or mask, and the snoring time occurring during the treatment.

The OSAS treatment consists in having the patient breath at a relative pressure, that can vary from 4 to 20 cm H₂O, prescribed by the doctor. This pressure is regulated by the air generator and corresponds substantially to that in the mask connected to the nose of the patient to within the tolerance of the pressure drop in the patient circuit.

The existing venturi tubes have, for the flow rate band considered, between 0 and 130 1/min, a pressure loss, also called head loss, between the inlet and the outlet of the venturi tube that is greater than 1 cm H₂O. This value is very high. In practice, the additional head loss generated by an apparatus inserted into the patient circuit that can be accepted is at most 0.2 cm H₂O in use with no unintentional leak at the mask, that is to say for a gas flow rate of between 60 and 70 l/min. That corresponds to 0.8 cm H₂O for a flow rate of 130 l/min.

The venturi tube 16 is designed in such a way as to obtain, for the flow rate band considered, a pressure loss lower than 80 Pa. To achieve this performance level, the venturi tube 16 is, preferably, a nozzle venturi having a geometry as represented in FIG. 3.

The venturi tube 16 comprises a cylindrical inlet 40 and outlet 42 that have the same diameter D1. The diameter D1 is adapted to the outlet diameter of the air generator of the treatment apparatus 6. It is notably equal to 17.3 mm.

The first pressure sensor 101 of the venturi tube is positioned at the inlet 40 to measure the pressure of the gas at the inlet of the venturi tube.

The venturi tube 16 also comprises a convergent 44, having an inlet diameter D3 and formed by two circular arcs 46, 48 of respective radii R1, R2, as appears clearly in the enlarged view of the circular arcs in FIG. 3. The centers of the circular arcs 46,48 are identified relative to the inlet 40 of the venturi tube 16 on the x axis and relative to the axis of the venturi tube on the y axis.

According to a preferred embodiment, the center of the first circular arc is positioned at (x1,y1)=(2 mm; 7.5 mm) and R1=2 mm.

Furthermore, preferably, the center of the second circular arc is positioned at (x2,y2)=(3 mm; 8.3 mm) and R2=3.3mm.

Preferably, the inlet diameter D3 of the convergent 44 is chosen to be between 14 and 16 mm, notably equal to 15 mm.

The venturi tube 16 also comprises a cylindrical neck 50 of diameter D2 and extending over a length L2. The length L2 is chosen to be between 5 and 10 mm, preferably equal to 7.5 mm. The diameter D2 of the neck 50 is preferably between 9 and 11 mm, notably equal to 10 mm.

The second pressure sensor 102 of the venturi tube is positioned at the neck 50 to measure the pressure of the gas at said neck of the venturi tube.

The venturi tube 16 finally comprises a divergent 52, of tapered form, situated between the neck 50 and the outlet 42. The divergent 52 is characterized by an angle of divergence 2α relative to the horizontal axis x (the angle α of FIG. 3 representing half the angle of divergence).

The angle of divergence 2α is preferably between 5 and 15°.

The connecting part between the neck 50 and the divergent 52 is a sharp angle.

Various nozzle venturis have been tested in the context of the present invention.

A first venturi tube is characterized by a neck diameter D2 equal to 10 mm and an angle of divergence 2α equal to 10°.

A second venturi tube is characterized by a neck diameter D2 equal to 10 mm and an angle of divergence 2α equal to 8°.

These venturi tubes have very satisfactory performance levels since they obtain a total head loss between the inlet 40 and the outlet 42 of said venturi tube that is limited to 68 Pa for a flow rate of 130 liters per minute.

Moreover, by defining the sensitivity of the venturi tube for detecting breathing events as being the ratio between the useful differential pressure (that is to say, the difference between the pressure measured by the sensor 101 at the plane P2 situated upstream of the inlet of the neck and the pressure measured by the sensor 102 at the plane P5 situated in the neck) and the pressure loss of the gas between the inlet 40 and the outlet 42 of said venturi tube, the following results are obtained:
the first venturi tube has a sensitivity equal to 6.26; and
the second venture tube has a sensitivity equal to 6.36.

Both of them therefore exhibit both head losses and a flow rate measurement accuracy that are in accordance with the functional requirements of the apparatus.

Preferred dimensions for the venturi tube 16 are summarized in the table below.

TABLE

| Angle | D1 | D2 | L2 | D3 | R1 | x1 | y1 | R2 | x2 | y2 |
|---|---|---|---|---|---|---|---|---|---|---|
| α | (mm) | (mm) | (mm) | (mm) | (mm) | (mm) | (mm) | (mm) | (mm) | (mm) |
| 4° | 17.3 | 10 | 7.5 | 15 | 2 | 2 | 7.5 | 3.3 | 3 | 8.3 |

The apparatus for monitoring the compliance with a treatment for obstructive sleep apnea of the present invention is particularly well suited to monitoring compliance with a treatment for obstructive sleep apnea in a patient.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. An apparatus (2, 4) for monitoring a treatment for obstructive sleep apnea comprising a gas passage (10) and a venturi tube (16) having a cylindrical inlet (40) and outlet (42), said venturi tube (16) being arranged axially in said gas passage (10), a first pressure sensor (101) and a second pressure sensor (102), characterized in that
the diameters (D1) of the inlet (40) and the outlet (42) of the venturi tube (16) are between 10 and 25 mm, and
the venturi tube (16) comprises, arranged in succession between said inlet (40) and outlet (42), a convergent portion (44) in the form of a circular arc (46, 48), a cylindrical neck (50) with a diameter smaller than the diameter (D1) of the inlet (40), and a divergent portion (52) characterized by an angle of divergence (α) of between 5° and 15°,
and in which the diameter (D2) of the neck (50) of the venturi tube (16) is between 5 and 15 mm and the length (L2) of the neck (50) of the venturi tube (16) is between 3 and 12 mm.

2. The apparatus as claimed in claim 1, characterized in that the venturi tube (16) is configured to have a pressure loss, between its inlet (40) and its outlet (42), less than 100 Pa for a gas flow rate in the passage (10) of between 110 an 150 l/min.

3. The apparatus as claimed in claim 1, characterized in that inlet (40) and the outlet (42) of the venturi tube (16) have the same diameter (D1).

4. The apparatus as claimed in claim 3, characterized in that the inlet (40) and the outlet (42) have an identical diameter of between 13 and 20 mm, preferably between 16 and 18 mm.

5. The apparatus as claimed in claim 1, characterized in that the venturi tube (16) comprises a convergent portion (44) having a profile formed by two successive circular arcs (46,48).

6. The apparatus as claimed in claim 1, characterized in that the diameter (D2) of the neck (50) is between 7 and 13 mm.

7. The apparatus as claimed in claim 1, characterized in that the angle of divergence is less than or equal to 12°.

8. The apparatus as claimed in claim 1, characterized in that the length (L2) of the neck (50) is between 5 and 10 mm.

9. The apparatus as claimed in claim 1, characterized in that the venturi tube (16) comprises a convergent portion (44) having a profile formed by a first circular arc (46) of first radius (R1) and a second circular arc (48) of second radius (R2) in which said first and second radii are less than 10 mm.

10. The apparatus as claimed in claim 1, characterized in that the venturi tube (16) has a total length of between 90 and 100 mm.

11. The apparatus as claimed in one of the preceding claims claim 1, characterized in that it also comprises:
a first pressure sensor (101) arranged at the inlet (40) of the venturi tube (16) to measure a pressure of a gas at the inlet (40) of the venturi tube (16); and
a second pressure sensor (102) arranged at the neck (50) of the venturi tube (16) to measure a pressure of the gas at the neck (50) of the venturi tube (16).

12. The apparatus as claimed in claim 11, characterized in that it also comprises:
a data processing device (22) configured to process the flow rate and pressure values measured by the first and second pressure sensors (101, 102) to deduce therefrom at least one treatment duration datum and at least one treatment effectiveness datum;
a data storage (24) arranged to store at least one of said data; and
a transmission device (26) arranged to transmit at least one of said data to a remote server (34).

13. The apparatus as claimed in claim 1, in which the venturi tube (16) is arranged and/or configured to have a useful differential pressure approximately 6 times greater than the pressure loss of a gas between the inlet (40) and the outlet (42) of said venturi tube (16).

14. A sleep apnea treatment installation comprising a pressurized gas source (6) linked to a breathing mask (8) via a gas duct (30), characterized in that the sleep apnea treatment installation comprises an apparatus (2, 4) as claimed in claim 1, operably arranged between said gas source (6) and said breathing mask (8).

15. The installation as claimed in claim 14, characterized in that the pressurized gas source (6) is a device of CPAP or BiPAP type.
